(19) United States
(12) Patent Application Publication
(10) Pub. No.: US 2019/0015316 A1
(43) Pub. Date: Jan. 17, 2019

(54) STYLING AGENTS WITH IMPROVED MOISTURE RESISTANCE AND WASHABILITY

(71) Applicant: Henkel AG & Co. KGaA, Duesseldorf (DE)

(72) Inventors: Thorsten Knappe, Schenefeld (DE); Tim Bethge, Hamburg (DE); Thea Kroehnke, Hamburg (DE)

(73) Assignee: Henkel AG & Co. KGaA, Duesseldorf (DE)

(21) Appl. No.: 16/021,880

(22) Filed: Jun. 28, 2018

(30) Foreign Application Priority Data
Jul. 11, 2017 (DE) 10 2017 211 854.7

(51) Int. Cl. A61K 8/891; A61K 8/81; A61K 8/37; A61K 8/19; A61Q 5/06

(52) U.S. Cl. CPC A61K 8/891; A61K 8/8147; A61K 8/8152; A61K 8/8158; A61K 2800/5424; A61K 8/19; A61Q 5/06; A61K 2800/87; A61K 8/37

(57) ABSTRACT

The present disclosure relates to cosmetic agents for temporary deformation of keratinous fibers, particularly human hair, which contain completely non-neutralized anionic polymer and at least one special ester and/or siloxane compound. These cosmetic agents have extremely good moisture resistance and washability. The present disclosure also relates to the use of these cosmetic agents and a method for temporary deformation of keratinous fibers using these agents.

### CROSS-REFERENCE TO RELATED APPLICATION

This application claims priority to German Patent Application No. 10 2017 211 854.7, filed Jul. 11, 2017, which is incorporated herein by reference in its entirety.

### TECHNICAL FIELD

The application relates to the technical field of temporary shaping of keratinous fibers, more particularly human hair. The subject of the patent is cosmetic agents containing at least one neutralizing anionic polymer based on acrylic acid and acrylates, as well as at least one ester and/or at least one linear siloxane. An additional subject of the present application is a cosmetic product containing an application device with a spray valve and a cosmetic agent as contemplated herein. A final subject of the present disclosure is methods for temporary deformation of keratinous fibers using said agents and the use of special esters and/or linear siloxanes in styling agents to improve the moisture resistance and washability of said agents.

### BACKGROUND

An attractive hairstyle is generally considered an indispensable part of a well-groomed appearance. Current fashion trends favor hairstyles which can only be formed using firming active ingredients and/or can be maintained for a longer period of up to several days. Therefore, hair treatment agents that permanently or temporarily shape the hair play an important role. While permanent deformation will modify the keratinous fibers due to reduction and oxidation, such modifications of the chemical structure do not take place with temporary deformation. Corresponding agents for temporary deformation normally contain synthetic polymers and/or waxes as a firming active ingredient.

The most important feature of an agent for temporary deformation of keratinous fibers, also referred to hereinafter as a styling agent, of giving the treated fibers in the modeled shape—i.e. a shape impressed on the hair—the strongest possible hold. If the keratinous fibers are human hair, this is also referred to as a strong hairstyle hold or a high degree of retention of the styling agent. The hairstyle hold is essentially determined by the type and amount of firming active ingredients used, wherein the other components of the styling agent and application form can also have an influence.

In addition to a high degree of hold, the styling agent must fulfill a series of additional requirements. They can be roughly divided in to characteristics on the hair, characteristics of the respective formulation, e.g. the sprayed aerosol or non-aerosol, and characteristics relating to the handling of the styling agent, wherein the special importance is given to the characteristics on the hair. This includes, in particular, moisture resistance, low stickiness and a balanced conditioning effect. Furthermore, a styling agent should be universally applicable for all hair types, insofar as possible, and mild to the hair and skin.

In order to satisfy the various requirements, a multitude of synthetic polymers was developed in the prior art as firming active ingredients, which can be used in styling agents. The polymers can be divided into cationic, anionic, nonionic and amphoteric firming polymers. Ideally, when applied on the hair, the polymers produce a polymer film which gives the hairstyle a strong hold, yet remains adequately flexible in order to not break under stress and can be easily washed out of the hair. If the polymer film is too brittle, so-called flakes or residues form, which detach during movement of the hair and give the impression that the user of the corresponding styling agent has dandruff. If the polymer film cannot be washed out or only partially washed out, residues remaining on the hair can lead to an unpleasant sensation or can make it difficult to reform the hair.

The polymer films obtained with synthetic polymers and polymer combinations known in the prior art have a satisfactory hold, but the hairstyle hold achieved in this connection can diminish considerably in a humid environment and the hair can appear to be adhesively bonded and unnatural. However, this diminished hold in a humid environment does not correspond to the consumer's requirement for a consistently high long-term hold independently of external weather influences. Therefore, there is a need to improve the moisture resistance of polymer films obtained with polymers and polymer combinations, but without negatively influence the listed characteristics of cosmetic agents, particularly the washability.

Therefore, the present disclosure addressed the problem of providing a styling agent which provides a long-term hold, high flexibility and low stickiness, as well as outstanding moisture resistance—particularly resistance to sweat and water—and good washability. Furthermore, these agents should be stable during storage and can be easily and uniformly distributed on the fibers and enable complete emptying of the container.

Surprisingly, it has now been found that outstanding moisture resistance can be achieved with the addition of the special ester and/or siloxane to produce a completely non-neutralized anionic polymer based on acrylic acid and acrylates, but without negatively influencing the hold, flexibility and washability. These agents also have high stability during storage and can be distributed uniformly on the fibers. Furthermore, the addition of the ester and/or siloxane does not lead to premature clogging of the removal opening, so that complete emptying of the cosmetic agent from the storage containers is possible.

### BRIEF SUMMARY

This disclosure provides a cosmetic agent for temporary deformation of keratinous fibers. The agent includes

a) at least one anionic copolymer comprising at least one structural unit of the formula (I) and at least one structural unit of the formula (II) and at least one structural unit of the formula (III)

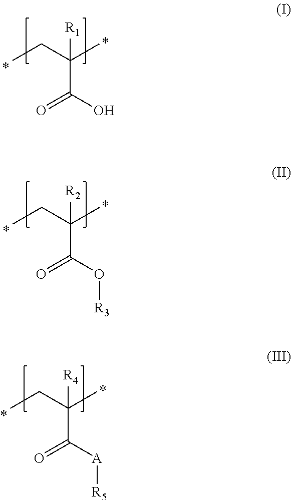

wherein

R₁, R₂, R₄ each independently denote a hydrogen atom or a C₁-C₄-alkyl group,

R₃ denotes a branched or unbranched, saturated or unsaturated C₁-C₁₂-alkyl group,

R₅ denotes a branched or unbranched, saturated or unsaturated C₆-C₁₄-alkyl group,

A denotes oxygen, sulfur or a NH group,

b) at least one ester of the formula (IV) and/or at least one siloxane of the formula (V),

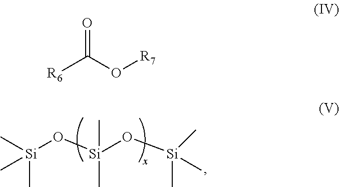

wherein

R₆ denotes a branched or unbranched, saturated or unsaturated C₆-C₃O-alkyl group,

R₇ denotes a branched or unbranched, saturated or unsaturated C₁-C₁₂-alkyl group, and

x denotes integers from about 0 to about 8, and

c) about 0 wt. % of alkalizing agents relative to the total weight of the cosmetic agent.

This disclosure also provides a cosmetic product that includes

a) an application device with a spray valve,

b) the cosmetic agent disposed in the application device, and

c) a propellant in a total amount of from about 0 to about 95 wt. % relative to the total weight of the agent in the application device.

This disclosure further provides a method for temporary deformation of keratinous fibers. The method includes the steps of:

a) preparing a cosmetic agent,

b) applying the cosmetic agent prepared in step a),

c) distributing the cosmetic agent applied in step b) on the keratinous fibers resulting in the deformation of the keratinous fibers in a desired shape.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Therefore, a first subject of the present disclosure is a cosmetic agent for temporary deformation of keratinous fibers containing

a) at least one anionic copolymer comprising at least one structural unit of the formula (I) and at least one structural unit of the formula (II) and at least one structural unit of the formula (III)

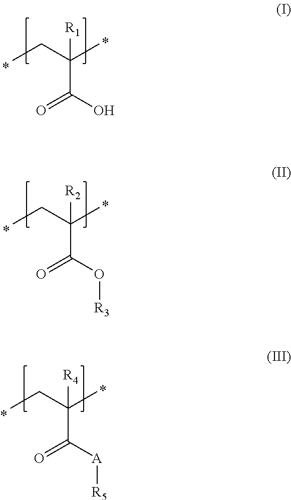

wherein
R₁, R₂, R₄ each independently denote a hydrogen atom or a C₁-C₄-alkyl group,
R₃ denotes a branched or unbranched, saturated or unsaturated C₁-C₁₂-alkyl group,
R₅ denotes a branched or unbranched, saturated or unsaturated C₆-C₁₄-alkyl group,
A denotes oxygen, sulfur or a NH group,
b) at least one ester of the formula (IV) and/or at least one siloxane of the formula (V),

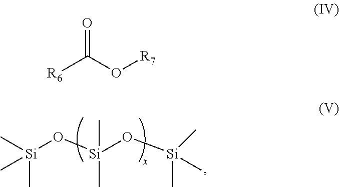

wherein
R₆ denotes a branched or unbranched, saturated or unsaturated C₆-C₃₀-alkyl group,
R₇ denotes a branched or unbranched, saturated or unsaturated C₁-C₁₂-alkyl group,
and x denotes integers from about 0 to about 8, and
c) about 0 wt. % of alkalizing agents relative to the total weight of the cosmetic agent.

With the addition of esters of the formula (IV) and/or siloxanes of the formula (V) to produce a completely non-neutralized anionic polymer based on acrylic acid and acrylates, an improved moisture resistance of the cosmetic agents is achieved. Therefore, the cosmetic agents promote a permanent and consistently high hairstyle hold even in even in changing external environmental influences. Furthermore, use of the ester and/or siloxane leads to improved washability of said agents and, in this manner, promotes reliability and complete removal of said agents during the cleaning of the hair. In addition, use of the ester and/or siloxane does not lead to a negative influence on the storage stability, the spray pattern or the complete emptying of the cosmetic agents from the storage containers.

According to the above formulas, a chemical bond bearing the symbol “*” denotes a free valence of the corresponding structural fragment. A free valence is the number of atomic bonds emanating from the corresponding structural fragment at the position bearing the symbol “*”. According to the present disclosure, an atomic bond bearing the symbol preferably emanates from the structural fragments from the position bearing the symbol “*” to form other structural fragments.

The term keratinous fibers is basically understood to mean all animal hair, such as wool, horse hair, mohair, fur, feathers and products or textiles produced therefrom. However, the keratinous fibers are preferably human hair.

In the context of the present disclosure, the term “anionic polymers” is understood to mean such polymers which have at least one structural unit with permanently anionic groups in a protic solvent under standard conditions, wherein the anionic groups must be compensated while maintaining the electrical neutrality. as contemplated herein, said anionic groups include carboxyl groups, in particular.

Furthermore, the term “alkalizing agents” in the context of the present disclosure is understood to mean compounds which are capable of absorbing at least one proton (H+) and/or neutralize an acid while producing a salt. This is understood to mean alkaline and earth alkaline hydroxides, ammonia and organic amines, such as alkanolamines.

The term “fatty acids” as used in the scope of the present disclosure should be understood as aliphatic carboxylic acids having unbranched or branched carbon radicals with from 4 to 40 carbon atoms. The fatty acids used in the scope of the present disclosure can be naturally occurring and synthetically manufactured fatty acids. Furthermore, the fatty acids can be mono- or polyunsaturated.

Finally, the term “fatty alcohol” in the scope of the present disclosure should be understood as, aliphatic, monovalent, primary alcohols having unbranched or branched hydrocarbon radicals with from 4 to 40 carbon atoms. The fatty alcohols used in the scope of the present disclosure can also be mono- or polyunsaturated.

Unless otherwise specified, the wt. % presently relates to the total weight of the cosmetic agents, wherein the sum of all ingredients of the inventive agents amounts to about 100 wt. %. If the inventive cosmetic agents contain at least one propellant, the specification wt. % is relative to the total weight of the cosmetic agent, including the propellant.

As a first essential component a), the cosmetic agent contains at least one anionic polymer which contains at least one structural unit of formulae (I) to (III). The radicals R₁, R₂ and R₄ in the structural units of formulae (I) to (III) can denote C₁-C₄-alkyl groups. Examples of such groups are methyl-, ethyl-, propyl-, isopropyl-, hydroxypropyl-, butyl-, sec-butyl-, isobutyl-, tert-butyl- and hydroxy butyl groups. Furthermore, the radicals R₃ and R₅ in the structural units of formulae (II) and (III) denote C₁-C₁₂- and C₆-C₁₄-alkyl groups. Such groups include pentyl-, hexyl-, heptyl-, capryl-, caprin-, lauryl- and myristyl groups.

In the context of the present disclosure, it is preferred that the radicals R₁, R₂ and R₄ denote specific groups in the formulae (I) to (III). Therefore, preferred embodiments of this subject of the present disclosure are exemplified in that the radicals R₁ and R₄ in the structural units of the formulae (I) and (III) each independently denote a hydrogen atom and that the radical R₂ in the structural unit of the formula (II) denotes a methyl group. Therefore, preference is given the use of anionic polymers based on acrylic acids, methacrylates and acrylamides and/or acrylates. The use of such anionic polymers in combination with the ester of the formula (IV) and/or the siloxane of the formula (V) leads to an especially high moisture resistance and washability of the cosmetic agent.

Moreover, in the context of the present disclosure, it has been found to be advantageous if the radical R₃ in the structural unit of the formula (II) denotes specific groups. Therefore, it is preferred that the radical R₃ in the structural unit of the formula (II) denotes a branched C₃-C₆-alkyl group, particularly a *—CH₂—C(CH₃)₂ group. In the process, the * symbol indicates the linking of the radical R₃ with the oxygen atom of the structural unit of the formula (II). The radical R₃, therefore, is bound to the carbonyl group of the structural unit of the formula (II) by the CH₂ group. The use of anionic polymers, which contains branched methacrylates, in particular, has been found, in combination with the ester of the formula (IV) and/or the siloxane compound of the formula (V), to be particularly advantageous in regard to moisture resistance and improved washability of the polymer film.

Furthermore, it is preferred as contemplated herein that A in the structural unit of the formula (III) denotes a NH group. Therefore, preferred anionic polymers contain at least one structural unit based on acrylamides. The use of anionic polymers based on acrylamides, in combination with the component b), leads to improved moisture resistance, however, without negatively influencing the washability.

Furthermore, in the context of the present disclosure, it has been found to be advantageous if the radical R₅ in the structural unit of the formula (III) denotes specific groups. Therefore, as contemplated herein, it is preferred that the radical R₅ in the structural unit of the formula (III) denotes a branched C₆-C₁₀-alkyl group, particularly a *—C(CH₃)₂—CH₂—C(CH₃)₃ group. In the process, the * symbol indicates the linking of the radical R₅ with the unit A of the structural unit of the formula (III). The use of anionic polymers, which contains branched acrylamides and acrylates, in particular, has been found, in combination with the ester of the formula (IV) and/or the siloxane compound of the formula (V), to be particularly advantageous in regard to the improved moisture resistance and washability of the polymer film.

as contemplated herein, therefore, it is particularly advantageous to use of anionic polymers which contain a structural unit of the formula (I), at least one structural unit of the formula (II) and at least one structural unit of the formula (III)

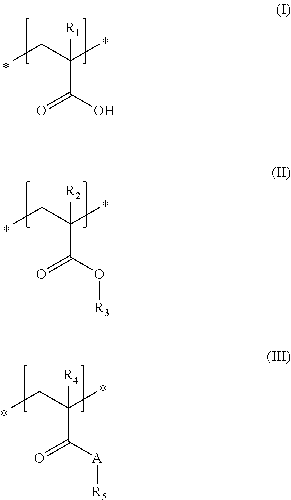

wherein
R₁ and R₄ each denote a hydrogen atom,
R₂ denotes a methyl group,
R₃ denotes a *—CH₂—C(CH₃)₂ group,
R₅ denotes a *—C(CH₃)₂—CH₂—C(CH₃)₃ group and
A denotes a NH group,

The use of such anionic polymers based on acrylic acid, 2-methylpropylmethacrylate and octylacrylamide in combination with at least one ester of the formula (IV) and/or a siloxane of the formula (V), it has been found to be particularly advantages in regard to moisture resistance and the washability of the cosmetic agents. Therefore, the use of such polymers results in a high a flexible hairstyle hold which does not become adhesive under the influence of moisture and thus give an unnatural impression.

Preferably used anionic polymers have specific average molecular weights M_{w}. Determination of these molecular weights can take place, for example, by coupling gel permeation chromatography (GPC) to a Fourier transform mass spectrometer (FTMS), as described in Aaserud D. J et. al; “Gel Permeation Chromatography Coupled to Fourier Transform Mass Spectrometry for Polymer Characterization”; Anal. Chem. 1999, 71, 4793-4799. Therefore, preferred embodiments of this subject matter are exemplified in that the anionic polymer has a mean molecular weight M_{w} of from about 50,000 to about 250,000 g/mol, preferably from about 80,000 to about 220,000 g/mol, more preferably from about 100,000 to about 200,000 g/mol, particularly from about 110,000 to about 180,000 g/mol.

Cosmetic agents preferred as contemplated herein contain the at least one anionic polymer a) in a total amount of from about 1.0 to about 8.0 wt. %, preferably from about 1.5 to about 6.0 wt. %, more preferably from about 2.0 to about 5.5 wt. %, most preferably from about 2.5 to about 5.0 wt. %, relative to the total weight of the cosmetic agent. Use of these amounts of anionic polymer results in a high and flexible hairstyle hold, but without negatively influencing the stability in storage, the usability, particularly the spray pattern, or the complete emptying of the storage containers.

As a second essential component b), the cosmetic agent contains at least one ester of the formula (IV) and/or at least one siloxane of the formula (V). The radicals R₆ and R₇ in the structural units of the formula (IV) can denote C₁-C₃O-alkyl groups. Examples of such groups are methyl-, ethyl-, propyl-, isopropyl-, hydroxpropyl-, butyl-, sec-butyl-, isobutyl-, tert-butyl-, hydroxybutyl-, pentyl-, hexyl-, capryl-, caprin-, lauryl-, myristyl-, cetyl-, stearyl-, arachidyl-, behenyl-, oyel- and ceryl groups.

Furthermore, in the context of the present disclosure, it has been found to be advantageous if the radicals R₆ and R₇ in the formula (IV) denote specific radicals. Advantageous embodiments of this subject of the present disclosure are thus exemplified in that the radical R₆ in the formula (IV) denotes a linear C₁₀-C₂₀ alkyl group, particularly a linear C₁₃ alkyl group, and the radical R₇ denotes a branched C₂-C₄ alkyl group, particularly an isopropyl group. Therefore, a particularly preferred ester of the formula (IV) is isopropyl myristate. The use of isopropyl myristate in combination with the anionic polymer, particularly the anionic polymer based on acrylic acid, 2-methylpropyl methacrylate and octylacrylamide, produces an especially good moisture resistance and washability of the cosmetic agents. Moreover, the use of this special ester of the formula (IV) does not have a negative influence on the hold, storage stability and the application characteristics of the cosmetic agents.

Moreover, it has been found to be preferable as contemplated herein that a special siloxane of the formula (V) is used in addition to or instead of the ester of the formula (IV), particularly isopropyl myristate. Therefore, in the context of the present disclosure, it is advantageous if x in the formula (V) denotes the integer 1. Therefore, it is particularly preferred that the cosmetic agent contains disilyloxysilane (also called trisiloxane). The use of disilyloxysilane in combination with the anionic polymer, particularly the anionic polymer based on acrylic acid, 2-methylpropyl methacrylate and octylacrylamide, produces an especially good moisture resistance and washability of the cosmetic agents. Moreover, the use of this special siloxane of the formula (V) does not have a negative influence on the hold, stability under storage and the application characteristics of the cosmetic agents.

Therefore, particular preference is given to the use of isopropyl myristate and/or disilyloxysilane in the cosmetic agent b). The use of a combination of isopropyl myristate and disilyloxysilane has been found to be particularly advantageous in regard to the moisture resistance and washability, however, without negatively influencing the hold, the storage stability or the application characteristics of the cosmetic agents.

Therefore, a particularly preferred embodiment in the context of the first subject of the present disclosure is cosmetic agents for temporary deformation of keratinous fibers containing

a) at least one anionic copolymer comprising at least one structural unit of the formula (I) and at least one structural unit of the formula (II) and at least one structural unit of the formula (III)

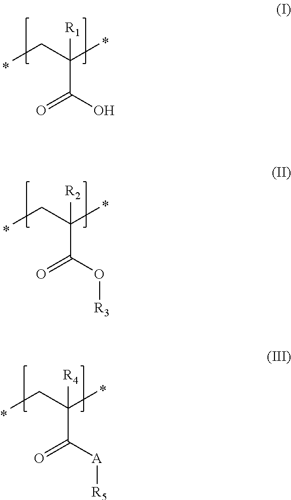

wherein
R₁ and R₄ each denote a hydrogen atom,
R₂ denotes a methyl group,
R₃ denotes a *—CH₂—C(CH₃)₂ group,
R₅ denotes a *—C(CH₃)₂—CH₂—C(CH₃)₃ group and
A denotes a NH group,
b) at least one ester of the formula (IV) and/or at least one siloxane of the formula (V),

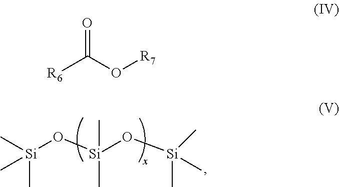

wherein
R₆ denotes a linear C₁₃-alkyl group,
R₇ denotes an isopropyl group, and
x denotes the integer 1,
c) about 0 wt. % of alkalizing agents relative to the total weight of the cosmetic agent.

Such cosmetic agents have outstanding hold and good moisture resistance and thus do not lead to adhesion of the hair due to the influence of moisture. Furthermore, with the addition of isopropyl myristate and/or trisiloxane, the washability of these agents can be improved without negatively influencing the good moisture resistance. Moreover, the use of isopropyl myristate and/or trisiloxane does not have a negative influence on the storage stability and the application characteristics of the cosmetic agents.

Preference is given to the at least one ester of the formula (IV), particularly isopropyl myristate, and/or the siloxane of the formula (V), particularly disilyloxysilane, in specific quantity ranges. Therefore, it is preferred as contemplated herein that the at least one ester of the formula (IV) and/or the at least one siloxane of the formula (V) is contained in a total amount of from about 0.1 to about 3.0 wt. %, preferably from about 0.1 to about 2.5 wt. %, more preferably from about 0.1 to about 2.0 wt. %, even more preferably from about 0.2 to about 1.0 wt. % and particularly from about 0.2 to about 0.7 wt. % relative to the total weight of the cosmetic agent. The amounts listed above relate to the total amounts of ester and/or siloxane. If a mixture of ester and siloxane is used, the specified amounts relate to the total amount of the mixture of ester and siloxane. The listed total amounts promote high moisture resistance and washability without negatively influencing the hold, flexibility, storage stability or the application characteristics of the cosmetic agents.

The cosmetic agents as contemplated herein do not contain any alkalizing agents. The anionic polymers used as component a) in the agents, therefore, is present in fully non-neutralized form. In particular, it has been found to be advantageous in the scope of the present disclosure that none of the alkalizing agents mentioned below are contained. Therefore, preferred embodiments of this subject of the present disclosure are exemplified in that the alkalizing agent is selected from the group of organic and inorganic bases, particularly alkanolamines, sodium hydroxide, potassium hydroxide, lithium hydroxide and mixtures thereof. Due to the absence of the aforementioned alkalizing agents in the cosmetic agents, the moisture resistance and the washability can be increased.

The cosmetic agents contain the anionic polymer a), the at least one ester of the formula (IV) and/or the at least one siloxane of the formula (V) and, optionally, additional ingredients in a cosmetically compatible carrier.

Preferred cosmetically compatible carriers are hydrous, alcoholic or hydrous-alcoholic media.

Particular preference is given to the use of anhydrous carriers. Therefore, as contemplated herein it is preferred that the cosmetic agent contains water in a total amount of from about 0 to about 0.5 wt. %, preferably from about 0 to about 0.4 wt. %, more preferably from about 0 to about 0.3 wt. %, particularly from about 0 to about 0.05 wt. %, relative to the total weight of the alcoholic composition. By using anhydrous cosmetic carriers, a matting of the hair with application of the cosmetic agents can be avoided. Therefore, the use of anhydrous cosmetic carriers, particularly anhydrous alcoholic cosmetic carriers, makes it possible to achieve strong hold and outstanding moisture resistance without negatively influencing the shine of the hair. Low alcohols having from about 1 to about 4 carbon atoms are normally used for cosmetic purpose, such as ethanol and isopropanol, can be used, in particular as alcohols.

The cosmetic agents preferably do not contain any additional film-forming and/or solidifying polymers in addition to the anionic polymer a). as contemplated herein, therefore, it is preferred that the cosmetic agent contains less than about 0.1 wt. %, preferably less than about 0.05 wt. %, more preferably less than about 0.01 wt. % and particularly about 0 wt. % of additional film-forming and/or solidifying polymers, relative to the total weight of the cosmetic agent. Film-forming and solidifying polymers are understood to mean such polymers which leave a continuous film on the skin, the hair or the nails when drying. Particular preference is given to polymers which are adequately soluble in water or water/alcohol mixtures in order to be provided in a completely dissolved form in the cosmetic agents. The film-forming polymers can be of synthetic or natural origin. Film-forming polymers are also understood to mean such polymers which are capable of depositing a transparent polymer film on the hair when used in about 0.01 to about 20 wt. % hydrous, alcoholic or hydrous-alcoholic solution.

The cosmetic agents of the present disclosure can be packaged in the conventional forms for temporary deformation of keratinous fibers, particularly hair, such as hair gel, hairspray, mousse or hair wax. Packaging as hairspray is preferable.

Hairsprays require the presence of a propellant are packaged as so-called aerosols.

Therefore, preferred cosmetic agents as contemplated herein contain at least one propellant. as contemplated herein, therefore, it is preferred that the cosmetic agent contains at least one propellant in a total amount of from about 30 to about 70 wt. %, preferably from about 35 to about 75 wt. %, particularly from about 40 to about 60 wt. % relative to the total weight of the cosmetic agent. Suitable propellants as contemplated herein are selected from N20, dimethylether, CO2, air, alkanes with from about 3 to about 5 carbon atoms, such as propane, n-butane, iso-butane, n-pentane and iso-pentane, and the mixtures thereof. Particular preference is given to use of dimethyl ether as a propellant. However, the present disclosure also explicitly comprises the co-utilization of propellants of the type of fluorochloro hydrocarbons, more particularly however, the fluoro hydrocarbons.

The following tables list particularly preferred embodiments of AF 1 to AF 64 of the cosmetic agent as contemplated herein (all figures in wt. %).

| | AF 1 | AF 2 | AF 3 | AF 4 |
|---|---|---|---|---|
| Anionic polymer a) ¹⁾ | 1.0-8.0 | 1.5-6.0 | 2.0-5.5 | 2.5-5.0 |
| Ester and/or siloxane ₂₎ | 0.1-3.0 | 0.1-2.0 | 0.2-1.0 | 0.2-0.7 |
| Alkalizing agent | 0 | 0 | 0 | 0 |
| Cosmetic carrier | ad 100 | ad 100 | ad 100 | ad 100 |
| | AF 5 | AF 6 | AF 7 | AF 8 |
| Anionic polymer a) ³⁾ | 1.0-8.0 | 1.5-6.0 | 2.0-5.5 | 2.5-5.0 |
| Ester and/or siloxane ₂₎ | 0.1-3.0 | 0.1-2.0 | 0.2-1.0 | 0.2-0.7 |
| Alkalizing agent | 0 | 0 | 0 | 0 |
| Cosmetic carrier | ad 100 | ad 100 | ad 100 | ad 100 |
| | AF 9 | AF 10 | AF 11 | AF 12 |
| Anionic polymer a) ¹⁾ | 1.0-8.0 | 1.5-6.0 | 2.0-5.5 | 2.5-5.0 |
| Ester and/or siloxane ₄₎ | 0.1-3.0 | 0.1-2.0 | 0.2-1.0 | 0.2-0.7 |
| Alkalizing agent | 0 | 0 | 0 | 0 |
| Cosmetic carrier | ad 100 | ad 100 | ad 100 | ad 100 |
| | AF 13 | AF 14 | AF 15 | AF 16 |
| Anionic polymer a) ³⁾ | 1.0-8.0 | 1.5-6.0 | 2.0-5.5 | 2.5-5.0 |
| Ester and/or siloxane ₄₎ | 0.1-3.0 | 0.1-2.0 | 0.2-1.0 | 0.2-0.7 |
| Alkalizing agent | 0 | 0 | 0 | 0 |
| Cosmetic carrier | ad 100 | ad 100 | ad 100 | ad 100 |
| | AF 17 | AF 18 | AF 19 | AF 20 |
| Anionic polymer a) ¹⁾ | 1.0-8.0 | 1.5-6.0 | 2.0-5.5 | 2.5-5.0 |
| Ester and/or siloxane ₂₎ | 0.1-3.0 | 0.1-2.0 | 0.2-1.0 | 0.2-0.7 |
| Alkalizing agent ⁵⁾ | 0 | 0 | 0 | 0 |
| Cosmetic carrier | ad 100 | ad 100 | ad 100 | ad 100 |
| | AF 21 | AF 22 | AF 23 | AF 24 |
| Anionic polymer a) ³⁾ | 1.0-8.0 | 1.5-6.0 | 2.0-5.5 | 2.5-5.0 |
| Ester and/or siloxane ₂₎ | 0.1-3.0 | 0.1-2.0 | 0.2-1.0 | 0.2-0.7 |
| Alkalizing agent ⁵⁾ | 0 | 0 | 0 | 0 |
| Cosmetic carrier | ad 100 | ad 100 | ad 100 | ad 100 |
| | AF 25 | AF 26 | AF 27 | AF 28 |
| Anionic polymer a) ¹⁾ | 1.0-8.0 | 1.5-6.0 | 2.0-5.5 | 2.5-5.0 |
| Ester and/or siloxane ₄₎ | 0.1-3.0 | 0.1-2.0 | 0.2-1.0 | 0.2-0.7 |
| Alkalizing agent ⁵⁾ | 0 | 0 | 0 | 0 |
| Cosmetic carrier | ad 100 | ad 100 | ad 100 | ad 100 |
| | AF 29 | AF 30 | AF 31 | AF 32 |
| Anionic polymer a) ³⁾ | 1.0-8.0 | 1.5-6.0 | 2.0-5.5 | 2.5-5.0 |
| Ester and/or siloxane ₄₎ | 0.1-3.0 | 0.1-2.0 | 0.2-1.0 | 0.2-0.7 |
| Alkalizing agent ⁵⁾ | 0 | 0 | 0 | 0 |
| Cosmetic carrier | ad 100 | ad 100 | ad 100 | ad 100 |
| | AF 33 | AF 34 | AF 35 | AF 36 |
| Anionic polymer a) ¹⁾ | 1.0-8.0 | 1.5-6.0 | 2.0-5.5 | 2.5-5.0 |
| Ester and/or siloxane ₂₎ | 0.1-3.0 | 0.1-2.0 | 0.2-1.0 | 0.2-0.7 |
| Alkalizing agent | 0 | 0 | 0 | 0 |
| Propellant ⁶⁾ | 30-70 | 32-68 | 35-65 | 40-60 |
| Cosmetic carrier | ad 100 | ad 100 | ad 100 | ad 100 |
| | AF 37 | AF 38 | AF 39 | AF 40 |
| Anionic polymer a) ³⁾ | 1.0-8.0 | 1.5-6.0 | 2.0-5.5 | 2.5-5.0 |
| Ester and/or siloxane ₂₎ | 0.1-3.0 | 0.1-2.0 | 0.2-1.0 | 0.2-0.7 |
| Alkalizing agent | 0 | 0 | 0 | 0 |
| Propellant ⁶⁾ | 30-70 | 32-68 | 35-65 | 40-60 |
| Cosmetic carrier | ad 100 | ad 100 | ad 100 | ad 100 |
| | AF 41 | AF 42 | AF 43 | AF 44 |
| Anionic polymer a) ¹⁾ | 1.0-8.0 | 1.5-6.0 | 2.0-5.5 | 2.5-5.0 |
| Ester and/or siloxane ₄₎ | 0.1-3.0 | 0.1-2.0 | 0.2-1.0 | 0.2-0.7 |
| Alkalizing agent | 0 | 0 | 0 | 0 |
| Propellant ⁶⁾ | 30-70 | 32-68 | 35-65 | 40-60 |
| Cosmetic carrier | ad 100 | ad 100 | ad 100 | ad 100 |
| | AF 45 | AF 46 | AF 47 | AF 48 |
| Anionic polymer a) ³⁾ | 1.0-8.0 | 1.5-6.0 | 2.0-5.5 | 2.5-5.0 |
| Ester and/or siloxane ₄₎ | 0.1-3.0 | 0.1-2.0 | 0.2-1.0 | 0.2-0.7 |
| Alkalizing agent | 0 | 0 | 0 | 0 |
| Propellant ⁶⁾ | 30-70 | 32-68 | 35-65 | 40-60 |
| Cosmetic carrier | ad 100 | ad 100 | ad 100 | ad 100 |
| | AF 49 | AF 50 | AF 51 | AF 52 |
| Anionic polymer a) ¹⁾ | 1.0-8.0 | 1.5-6.0 | 2.0-5.5 | 2.5-5.0 |
| Ester and/or siloxane ₂₎ | 0.1-3.0 | 0.1-2.0 | 0.2-1.0 | 0.2-0.7 |
| Alkalizing agent ⁵⁾ | 0 | 0 | 0 | 0 |
| Propellant ⁶⁾ | 30-70 | 32-68 | 35-65 | 40-60 |
| Cosmetic carrier | ad 100 | ad 100 | ad 100 | ad 100 |
| | AF 53 | AF 54 | AF 55 | AF 56 |
| Anionic polymer a) ³⁾ | 1.0-8.0 | 1.5-6.0 | 2.0-5.5 | 2.5-5.0 |
| Ester and/or siloxane ₂₎ | 0.1-3.0 | 0.1-2.0 | 0.2-1.0 | 0.2-0.7 |
| Alkalizing agent ⁵⁾ | 0 | 0 | 0 | 0 |
| Propellant ⁶⁾ | 30-70 | 32-68 | 35-65 | 40-60 |
| Cosmetic carrier | ad 100 | ad 100 | ad 100 | ad 100 |
| | AF 57 | AF 58 | AF 59 | AF 60 |
| Anionic polymer a) ¹⁾ | 1.0-8.0 | 1.5-6.0 | 2.0-5.5 | 2.5-5.0 |
| Ester and/or siloxane ₄₎ | 0.1-3.0 | 0.1-2.0 | 0.2-1.0 | 0.2-0.7 |
| Alkalizing agent ⁵⁾ | 0 | 0 | 0 | 0 |
| Propellant ⁶⁾ | 30-70 | 32-68 | 35-65 | 40-60 |
| Cosmetic carrier | ad 100 | ad 100 | ad 100 | ad 100 |
| | AF 61 | AF 62 | AF 63 | AF 64 |
| Anionic polymer a) ³⁾ | 1.0-8.0 | 1.5-6.0 | 2.0-5.5 | 2.5-5-0 |
| Ester and/or siloxane ₄₎ | 0.1-3.0 | 0.1-2.0 | 0.2-1.0 | 0.2-0.7 |
| Alkalizing agent ⁵⁾ | 0 | 0 | 0 | 0 |
| Propellant ⁶⁾ | 30-70 | 32-68 | 35-65 | 40-60 |
| Cosmetic carrier | ad 100 | ad 100 | ad 100 | ad 100 |

¹⁾ containing structural units of formulae (I) to (III), where R₁, R₂ and R₄ = H or C₁—C₄-alkyl group, R₃ = branched or unbranched, saturated or unsaturated C₁—C₁₂-alkyl group, R₅ = branched or unbranched, saturated or unsaturated C₆—C₁₄-alkyl group, A = O, S or NH,
₂₎ ester of the formula (IV), where R₆ = branched or unbranched, saturated or unsaturated C₆—C₃₀-alkyl group, R₇ = branched or unbranched, saturated or unsaturated C₁—C₁₂-alkyl group; siloxane of the formula (V), where x = 0 to 8,
³⁾ containing structural units of formulae (I) to (III), where R₁, R₄ = H, R₂ = methyl group, R₃ = *—CH₂—C(CH₃)₂-group, R₅ = *—C(CH₃)₂—CH₂—C(CH₃)₃-group, A = NH
₄₎ ester of the formula (IV), where R₆ = linear C₁₃-alkyl group, R₇ = isopropyl group; siloxane of the formula (V), where x = 1,
⁵⁾ alkalizing agent selected from the group of organic and inorganic bases, particularly alkanolamines, sodium hydroxide, potassium hydroxide, lithium hydroxide and mixtures thereof,
⁶⁾ preferably selected from dimethyl ethers.

The use of the ester of the formula (IV) and/or the siloxane of the formula (V), particularly of isopropyl myristate and/or trisiloxane, in combination with the completely non-neutralized anionic polymer a), particularly with a non-neutralized anionic polymer based on acrylic acid, 2-methylpropyl acrylate and octylacrylamide, leads to an outstanding moisture resistance of the hairstyle and good washability of the cosmetic agents. Furthermore, no negative influence on the hold, the flexibility of the polymer film, the storage stability or the application characteristics of the cosmetic agents are observed.

Preferred embodiments AFT 1′ to AF 64′ correspond to the embodiments AF 1 to AF 64 listed above, however they only contain water in a total amount of from about 0 to about 0.5 wt. %, particularly from about 0 to about 0.05 wt. % relative to the total weight of the cosmetic agent. Due to the absence of water or the use of only extremely small amounts of water, a matting effect which occurs with addition of higher amounts of water can be avoided. Consequently, the use of these embodiments AF 1′ to AF 64′ does not lead to a reduced shine of the temporarily deformed hair due to the extremely low amounts of water and/or the absence of water. However, the absence of water or the use of extremely low amounts of water has no influence on the outstanding moisture resistance and washability with a simultaneously strong hold.

Further preferred embodiments AF 1″ to AF 64″ corresponds to the embodiments AF 1 to AF 64 or AF 1′ to AF 64′ listed above, however they contain less than about 0.1 wt. %, preferably less than about 0.05 wt. %, more preferably less than about 0.01 wt. %, particularly about 0 wt. % relative to the total weight of the cosmetic agent, further film-forming and/or solidfying polymers. Consequently, a negative influence on the moisture resistance of the anionic polymer a) is avoided.

Along with the components described above, the cosmetic agents contain additional ingredients. The group of these additional ingredients include, in particular, the cosmetically active adjuvants and additives, particularly additional nourishing agents.

For example, a silicone oil and/or a silicone gum which is different from the at least one siloxane compound of the formula (V) can be used as a nurturing ingredient. as contemplated herein, suitable silicone oils or silicone gums are, in particular dialkyl- and alkylarylsiloxanes, such as dimethylpolysiloxane and methylphenylpolysiloxane, and alkoxylated, quaternized or anionic derivatives thereof. Cyclical and linear polydialkylsiloxanes, alkoxylated and/or aminated derivatives thereof, dihydroxypolydimethylsiloxanes and polyphenylalkylsiloxanes, particularly PEG-12 dimethicone and PEG-14 dimethicone, are preferred.

The cosmetic agent can contain, for example, at least one protein hydrolysate and/or one of its derivatives as a nourishing agent. Protein hydrolysates are product mixtures obtained through the acidically, basically or enzymatically catalyzed decomposition of proteins. as contemplated herein, the term protein hydrolysates also includes total hydrolysates from individual amino acids and derivatives thereof, as well as mixtures of various amino acids. The molecular weight of the protein hydrolysates usable as contemplated herein is between from about 75, the molecular weight for glycine, and about 200,000, preferably from about 75 to about 50,000 and most preferably from about 75 to about 20,000 Dalton.

The cosmetic agent as contemplated herein can also contain, as a nourishing agent a vitamin, a provitamin, a vitamin precursor and/or derivatives thereof as contemplated herein, such vitamins, provitamins and vitamin precursors are usually assigned to the groups A, B, C, E, F and H.

The addition of panthenol can increase the flexibility of the polymer film formed with use of the cosmetic agent.

The cosmetic agent as contemplated herein can also contain, as a nourishing agent, a plant extract, as well as mono- and/or oligosaccharides and/or lipids.

Furthermore, oil bodies are suitable as care substances. The natural and synthetic cosmetic oil bodies include, for example, vegetable oils, liquid paraffin oils, isoparaffin oils and synthetic hydrocarbons, and di-n-alkyl ethers having a total of between 12 to 36 carbon atoms, in particular 12 to 24 carbon atoms.

Ester oils, i.e. esters of C₆ C₃₀ fatty acids with C₂ to about C₃₀ fatty alcohols, preferably monoesters of fatty acids with alcohols having from about 2 to about 24 carbon atoms, such as isopropyl myristate (Rilanit® IPM), isononanoic acid C₁₆-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are additional preferred nurturing oil bodies.

Furthermore, dicarboxylic acid esters, symmetrical, asymmetrical or cyclic esters of carbonic acid with fatty alcohols, trifatty acid esters of saturated and/or unsaturated linear and/or branched fatty acids with glycerol or fatty acid partial glycerides, which are to be understood as monoglycerides, diglycerides and technical mixtures thereof are suitable as care substances.

Furthermore, emulsifiers and/or surfactants are preferably contained in the cosmetic agents. PEG derivatives of hydrated castor oil are preferred, which are available, for example, under the name PEG Hydrogenated Castor Oil, such as PEG-30 Hydrogenated Castor Oil, PEG-33 Hydrogenated Castor Oil, PEG-35 Hydrogenated Castor Oil, PEG-36 Hydrogenated Castor Oil or PEG-40 Hydrogenated Castor Oil. As contemplated herein, use of PEG-40 Hydrogenated Castor Oil is preferred. These are preferably included in a total amount of from about 0.05 to about 1.5 wt. %, preferably from about 0.1 to about 1.0 wt. %, more preferably from about 0.2 to about 0.8 wt. %, particularly from about 0.3 to about 0.6 wt. %, relative to the total weight of the cosmetic agent.

The cosmetic agent is preferably packaged as a sprayable product, particularly in the form of a hairspray. Therefore, a further subject of the present disclosure is a cosmetic product, comprising

a) an application device with a spray valve,
b) a cosmetic agent in the application device, and
c) propellant in a total amount of from about 0 to about 95 wt. % relative to the total weight of the agent in the application device.

The total weight indicate above is relative to the sum of the weight of the cosmetic agent and the optionally available propellant.

According to a preferred embodiment of this subject, the application device is an aerosol container or a non-aerosol container. Therefore, preferred embodiments of this subject of the present disclosure are exemplified in that the application device is an aerosol or non-aerosol container, particularly an aerosol container. As contemplated herein, non-aerosol container is understood to mean a container under normal pressure with which the cosmetic agent inside the non-aerosol container is distributed as a spray mist by employing mechanical action, particularly a pump or squeeze system. The cosmetic agent in the form of a non-aerosol or spray mist preparation can be provided in any arbitrary propellant-gas-free spray system which has a dispensing container and a spray valve, such as a flexible pressure bottle with immersion tube and spray valve (squeeze bottle), in a balloon atomizer, which operates according to the Venturi principle or in a pump spray bottle having a pump lever which is actuated with the index finger or the entire hand as a type of trigger. In an embodiment of the cosmetic product in the form of a non-aerosol preferred for the cosmetic application, the dispensing container has a manually actuated spray pump.

By contrast an aerosol container in the context of the present disclosure is understood to mean a compressed gas container filled with propellant with which the cosmetic agent inside the aerosol container is distributed via a valve with the internal gas pressure of the aerosol container.

A particularly preferred embodiment of this subject of the present disclosure is cosmetic products in the form of hairsprays. In the context of the present disclosure, therefore, it is preferred that the cosmetic agent contains propellant in a total amount of from about 30 to about 70 wt. %, preferably from about 35 to about 75 wt. %, particularly from about 40 to about 60 wt. % relative to the total weight of the cosmetic agent. Particular preference is given to use of dimethyl ethers as a propellant.

Such cosmetic products can be produced in the normal manner. As a rule, all constituents of the cosmetic agent as contemplated herein are placed in a suitable pressure-resistant container. This is then closed by employing a valve. The desired amount of the special propellant is then added by employing conventional technology.

Vessels made of metal (aluminum, tinplate, tin), protected or splinter-proof plastic or of glass that is coated on the outside with plastic are eligible as pressure-proof containers, with compression strength, tensile strength, corrosion resistance, ease of filling, aesthetic aspects, portability, printability etc. being factors in their selection. Special inner coatings ensure corrosion resistance in relation to cosmetic agent contained in the pressure container. It is particularly preferred that the valves which are used have a valve paint coated on the interior, wherein the coating and valve material are compatible with each other. If aluminum valves are used, their valve heads can be coated on the inside with Micoflex varnish, for example. If tinsheet valves are used as contemplated herein, their valve heads can be coated on the inside with PET (polyethylene terephthalate), for example. With the quantity ratio of propellant to the other constituents of the preparations, the sizes of the aerosol droplets and the respective size distribution can be adjusted in the given spray device. The spraying rate of the aerosol spray is preferably from about 6.5 to about 10.0 g/10 s.

Particularly preferred cosmetic products in the form of an aerosol spray are packaged in an aerosol container with a stem-valve with a stem bore diameter of from about 0.27 to about 0.35 mm. Such valves are sold, for example, as valves of the type KE or type KEN from the Coster company.

However, the cosmetic agents as contemplated herein can also be packaged in a multi-chamber dispenser. The multi-chamber dispenser can be used so that one chamber is filled with the compressed propellant and another chamber is filled with the remaining constituents of the cosmetic agent. One such multi-chamber dispenser is, for example, a so-called bag-in-can package.

With respect to additional preferred embodiments of the method, particularly with regard to the cosmetic agent contained in this product, the statements made about the cosmetic agents as contemplated herein apply mutatis mutandis.

A further subject matter of the present disclosure is a method for temporary deformation of keratinous fibers, wherein the method comprises the following steps:

a) Preparation of a cosmetic agent or a cosmetic product,
b) application, particularly spraying, of the cosmetic agent or product prepared in step a),
c) distribution of the cosmetic agent or product applied in step b) on the keratinous fibers and deformation of keratinous fibers in the desired shape.

With regard to other preferred embodiments as contemplated herein, particularly with regard to the cosmetic agent or product used there, this applies mutatis mutandis to the statements made about the cosmetic agent as contemplated herein, as well as to the cosmetic product as contemplated herein.

An additional subject of the present disclosure is the use of an cosmetic agent or an cosmetic product for temporary deformation of keratinous fibers.

With regard to other preferred embodiments of the use as contemplated herein, particularly with regard to the cosmetic agent or product which is used, this applies mutatis mutandis to the statements made about the cosmetic agent as contemplated herein, as well as to the cosmetic product as contemplated herein.

Finally, an additional subject of the present disclosure is the use of an ester of the formula (IV) and/or a siloxane of the formula (V)

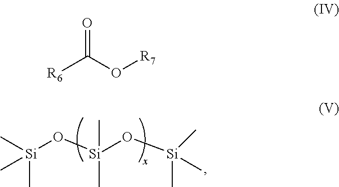

wherein
R₆ denotes a branched or unbranched, saturated or unsaturated C₆-C₃O-alkyl chain,
R₇ denotes a branched or unbranched, saturated or unsaturated C₁-C₁₂-alkyl chain, and
x denotes integers from about 0 to about 8, and in cosmetic agents for temporary deformation of keratinous fibers, for improvement of the washability of said agents out of keratinous fibers, particularly human hair.

According to a preferred embodiment of this subject of the present disclosure, the cosmetic agents contain fully non-neutralized anionic polymers comprising at least one structural unit of the formula (I) and at least one structural unit of the formula (II) and at least one structural unit of the formula (III)

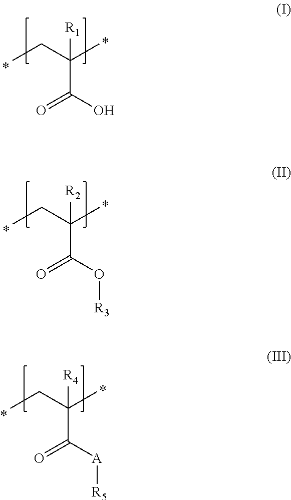

wherein
R₁, R₂ and R₄ each independently denote a hydrogen atom or a C₁-C₄-alkyl chain,
R₃ denotes a branched or unbranched, saturated or unsaturated C₁-C₁₂-alkyl chain,
R₅ denotes a branched or unbranched, saturated or unsaturated C₆-C₁₄-alkyl chain, and
A denotes oxygen, sulfur or a NH group.

Particular preference is given to use of the anionic polymers, esters of the formula (IV) and siloxane compounds of the formula (V), listed in connection with the cosmetic agent.

With respect to other preferred embodiments of the method according to the uses, the statements made regarding the agents as contemplated herein apply mutatis mutandis.

The following examples explain the present disclosure without limiting it:

### EXAMPLES

1. Formulations (all specifications in percent by weight, relative to the total weigh of the respective cosmetic agent):

| Raw material | V1 | V2 | E1 |
|---|---|---|---|
| Anionic polymer ¹⁾ | — | 3.5 | 3.5 |
| Amphomer LV 71 ²⁾ | 3.5 | — | — |
| AMP-Ultra PC 2000 ³⁾ | 0.61 | — | — |
| Esters of the formula (IV) ⁴⁾ | 0.05 | 0.60 | 0.60 |
| Siloxane compound of the formula (V) ⁵⁾ | — | 0.40 | 0.40 |
| Perfume | 0.2 | 0.2 | 0.2 |
| Ethanol 99%, denat. | — | — | 36.3 |
| Ethanol 96%, denat. | 36.4 | 36.3 | — |
| Water | 3.25 | — | — |
| Dimethyl ether | ad 100 | ad 100 | ad 100 |

¹⁾ containing structural units of formulae (I) to (III), where R₁, R₄ = H, R₂ = methyl group, R₃ = *—CH₂—C(CH₃)₂-group, R₅ = *—C(CH₃)₂—CH₂—C(CH₃)₃-group, A = NH,
²⁾ INCI name: octyl acrylamide/acrylates/butyl aminoethyl methacrylate copolymer (Akzo Nobel),
³⁾ INCI name: Aminomethyl propanol (Dow Corning),
⁴⁾ ester of the formula (IV), where R₆ = linear C₁₃-alkyl group, R₇ = isopropyl group,
⁵⁾ siloxane of the formula (V), where x = 1.

The cosmetic agents V1, V2 and E1 were obtained by mixing the ingredients above. The agents V1 and E1 were tested for their moisture resistance in the half-side test, wherein the same amounts of agents V1 and E1 were used in each case. Application of the agents took place on dry hair, wherein the agent V1 was applied on the right half of the head and the agent E1 was applied on the left half of the head. After the drying of the agents, the head was sprayed with water and the hair sensation was evaluated by 10 trained persons after drying again. In the process, the persons did not know which of the sides where the agent was applied. The application of water on the side of the head treated with the agent V1 led to an adhesive hairstyle with an unnatural sensation. By contrast, the use of water on the hair which was previously treated with the agent E1 resulted in a non-adhesive, natural hair sensation and a flexible hairstyle hold.

The use of a combination of a completely non-neutralized anionic polymer and an ester of the formula (IV) and a siloxane of the formula (V) leads to improved moisture resistance in comparison with cosmetic agents containing partially neutralized polymers.

The sides of the head treated with agents V1 and E1 were washed with a conventional hair shampoo and the hair sensation was evaluated by 10 trained persons to determine the washability. Both compositions could be removed without leaving residue and did not produce an unpleasant hair sensation. Therefore, the cosmetic agents led to increased moisture resistance of the hairstyle without negatively influencing the washability.

Composition V2 was compared with composition E1 in regard to the matting effect. For this purpose, the compositions were each sprayed on a watch glass and the dried polymer films were evaluated. In the case of composition V2, which contained water in an amount of 1.45 wt. %, a clouding of the polymer film was observed, whereas composition E1 produced a clear polymer film. Consequently, a matting effect on the hair can be avoided by reducing the amount of water in the agent.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic agent for temporary deformation of keratinous fibers, which comprises
a) at least one anionic copolymer comprising at least one structural unit of the formula (I) and at least one structural unit of the formula (II) and at least one structural unit of the formula (III) 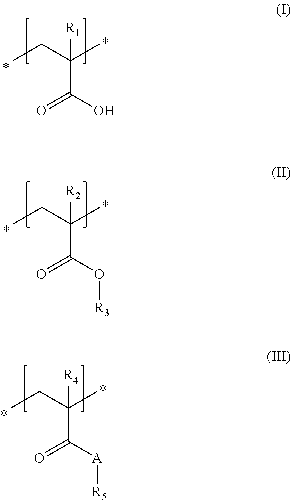
wherein
R₁, R₂, R₄ each independently denote a hydrogen atom or a C₁-C₄-alkyl group,
R₃ denotes a branched or unbranched, saturated or unsaturated C₁-C₁₂-alkyl group,
R₅ denotes a branched or unbranched, saturated or unsaturated C₆-C₁₄-alkyl group,
A denotes oxygen, sulfur or a NH group,
b) at least one ester of the formula (IV) and/or at least one siloxane of the formula (V),
wherein 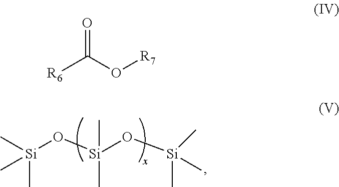
R₆ denotes a branched or unbranched, saturated or unsaturated C₆-C₃O-alkyl group,
R₇ denotes a branched or unbranched, saturated or unsaturated C₁-C₁₂-alkyl group, and
x denotes integers from about 0 to about 8, and
c) about 0 wt. % of alkalizing agents relative to the total weight of the cosmetic agent.

2. The cosmetic agent according to claim 1 wherein the radicals R₁ and R₄ in the structural units of the formulae (I) and (III) each independently denote a hydrogen atom and the radical R₂ in the structural unit of the formula (II) denotes a methyl group.

3. The cosmetic agent according to claim 1 wherein A in the structural unit of the formula (III) denotes a NH group.

4. The cosmetic agent according to claim 1 wherein the radical R₅ in the structural unit of the formula (III) denotes a branched C₆-C₁₀-alkyl group.

5. The cosmetic agent according to claim 1 wherein the at least one anionic copolymer a) is present in a total amount of from about 1.0 to about 8.0 wt. %, relative to the total weight of the cosmetic agent.

6. The cosmetic agent according to claim 1 wherein the at least one ester of the formula (IV) and/or the at least one siloxane of the formula (V) is included in a total amount of from about 0.1 to about 3.0 wt. %, relative to the total weight of the cosmetic agent.

7. The cosmetic agent according to claim 1 wherein the alkalizing agent is selected from the group of organic and inorganic bases, sodium hydroxide, potassium hydroxide, lithium hydroxide and mixtures thereof.

8. A cosmetic product, comprising
a) an application device with a spray valve,
b) the cosmetic agent as set forth in claim 1 disposed in the application device, and
c) propellant in a total amount of from about 0 to about 95 wt. % relative to the total weight of the agent in the application device.

9. A method for temporary deformation of keratinous fibers, wherein the method comprises:
a) preparing a cosmetic agent according to claim 1,
b) applying the cosmetic agent prepared in step a),
c) distributing the cosmetic agent applied in step b) on the keratinous fibers resulting in the deformation of the keratinous fibers in a desired shape.

10. (canceled)

11. The cosmetic agent according to claim 2 wherein A in the structural unit of the formula (III) denotes a NH group.

12. The cosmetic agent according to claim 2 wherein the radical R₅ in the structural unit of the formula (III) denotes a branched C₆-C₁₀-alkyl group.

13. The cosmetic agent according to claim 2 wherein the at least one anionic copolymer a) is present in a total amount of from about 1.0 to about 8.0 wt. %, relative to the total weight of the cosmetic agent.

14. The cosmetic agent according to claim 2 wherein the at least one ester of the formula (IV) and/or the at least one siloxane of the formula (V) is included in a total amount of from about 0.1 to about 3.0 wt. %, relative to the total weight of the cosmetic agent.

15. The cosmetic agent according to claim 2 wherein the alkalizing agent is selected from the group of organic and inorganic bases, sodium hydroxide, potassium hydroxide, lithium hydroxide and mixtures thereof.

16. The cosmetic agent according to claim 3 wherein the radical R₅ in the structural unit of the formula (III) denotes a branched C₆-C₁₀-alkyl group.

17. The cosmetic agent according to claim 3 wherein the at least one anionic copolymer a) is present in a total amount of from about 1.0 to about 8.0 wt. %, relative to the total weight of the cosmetic agent.

18. The cosmetic agent according to claim 3 wherein the at least one ester of the formula (IV) and/or the at least one siloxane of the formula (V) is included in a total amount of from about 0.1 to about 3.0 wt. %, relative to the total weight of the cosmetic agent.

19. The cosmetic agent according to claim 3 wherein the alkalizing agent is selected from the group of organic and inorganic bases, sodium hydroxide, potassium hydroxide, lithium hydroxide and mixtures thereof.

20. The cosmetic agent according to claim 1 wherein the at least one anionic copolymer a) is present in a total amount of from about 2.0 to about 5.5 wt. %, relative to the total weight of the cosmetic agent and wherein the at least one ester of the formula (IV) and/or the at least one siloxane of the formula (V) is included in a total amount of from about 0.2 to about 1.0 wt. %, relative to the total weight of the cosmetic agent.

21. The cosmetic agent according to claim 1 wherein the radicals R₁ and R₄ in the structural units of the formulae (I) and (III) each independently denote a hydrogen atom and the radical R₂ in the structural unit of the formula (II) denotes a methyl group,
wherein A in the structural unit of the formula (III) denotes a NH group,
wherein the radical R₅ in the structural unit of the formula (III) denotes a branched C₆-C₁₀-alkyl group,
wherein the at least one anionic copolymer a) is present in a total amount of from about 2.5 to about 5.0 wt. %, relative to the total weight of the cosmetic agent,
wherein the at least one ester of the formula (IV) and/or the at least one siloxane of the formula (V) is included in a total amount of from about 0.2 to about 0.7 wt. %, relative to the total weight of the cosmetic agent, and
wherein the alkalizing agent is selected from the group of organic and inorganic bases, sodium hydroxide, potassium hydroxide, lithium hydroxide and mixtures thereof.
